(19) United States
(12) Patent Application Publication
(10) Pub. No.: US 2020/0363423 A1
(43) Pub. Date: Nov. 19, 2020

(54) MISTRANSLATION METHOD FOR ASSESSING THE EFFECTS OF AMINO ACID SUBSTITUTIONS ON PROTEIN STABILITY AND FUNCTION

(71) Applicant: University of Washington, Seattle, WA (US)

(72) Inventors: Judit Villen, Seattle, WA (US); Ricard Rodriguez-Mias, Seattle, WA (US); Stanley Fields, Seattle, WA (US)

(73) Assignee: University of Washington, Seattle, WA (US)

(21) Appl. No.: 16/762,814

(22) PCT Filed: Nov. 9, 2019
(86) PCT No.: PCT/US2018/060077

§ 371 Date: May 8, 2020

Related U.S. Application Data
(60) Provisional application No. 62/583,986, filed on Nov. 9, 2017.

(51) Int. Cl. G01N 33/68

(52) U.S. Cl. CPC G01N 33/6803; G01N 2570/00

(57) ABSTRACT

This disclosure provides a non-genetic, high-throughput method of introducing and assaying effects of amino acid substitutions at a plurality of positions in a target protein. Mistranslation events are imposed stochastically during the translation of multiple copies of a target protein, resulting in a plurality of variants of the target protein (i.e., protein quasi-species). The analysis can be performed simultaneously for a plurality of different target proteins, including the entire proteome of a cell to produce a statistical proteome. The plurality of quasi-species or statistical proteome can be subjected to functional assays, such as enrichment or profiling assays, to determine the functional effect of the substitutions on the target protein(s).

### CROSS-REFERENCE(S) TO RELATED APPLICATION(S)

This application claims the benefit of U.S. Application No. 62/583,986, filed Nov. 9, 2017, the disclosure of which is hereby expressly incorporated by reference in its entirety herein.

### BACKGROUND

Recent innovations in DNA-sequencing methods have led to the discovery of millions of mutations that change the encoded protein sequences. However, the impact of a great majority of these mutations on protein function remains unknown. Current approaches addressing the effects of mutations are inadequate, as they typically rely on computational predictions whose accuracy is questionable. Alternative approaches interrogate only mutations individually or one protein at a time. Such approach are extremely time and resource intensive and would thus require hundreds of years to interpret existing mutational genetic data with current technology.

Accordingly, despite the great advances in the field of genetic sequencing and analysis there remains a need for efficient and effective strategies to assess the functional impact of observed mutations at the phenotypic (e.g., functional protein) level. The present disclosure addresses this and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a non-genetic, high-throughput method of introducing amino acid substitutions at a plurality of positions in a target protein and assaying the effects of such substitutions on the function of the target protein. The method comprises

(a) generating a plurality of variants of a target protein from the same reference mRNA sequence by stochastically introducing one or more amino acid substitutions during protein translation,

(b) applying the plurality of mistranslated variants to defined conditions in functional assays,

(c) measuring a value for the variants containing each amino acid substitution and a value for the variants that do not contain that amino acid substitution,

(d) comparing the value for the variants containing each amino acid substitution and the value for the variants that do not contain that amino acid substitution, and

(e) associating differences in the values with amino acid positions that are important for the structure or function of the target protein.

In some embodiments, step (c) specifically comprises measuring a value for each of the variants containing each amino acid substitution and a value for at least one variant that does not contain that amino acid substitution, and step (d) comprises comparing the values for the variants containing each amino acid substitution and the value for the variant that does not contain that amino acid substitution, and

In some embodiments, the method further comprises determining the presence of a substitution at one or more positions in each of the plurality of mistranslated variants. In some embodiments, the method further comprises compiling the differences in values determined in step (d) into a functional map of the target protein sequence. In some embodiments, the defined conditions in step (b) comprise a range of temperatures, pH values, chemical concentrations, or salt concentrations, and measuring a value comprises measuring the solubility profile of the plurality of mistranslated variants across the range. In some embodiments, the functional assay of step (b) comprises assaying subcellular localization. In some embodiments, the functional assay comprises assaying degradation of the plurality of mistranslated variants.

In another aspect, the disclosure provides a non-genetic, high-throughput method of assaying effects of amino acid substitutions at a plurality of positions in a target protein. The method comprises:

(a) generating a plurality of variants of a target protein from the same reference mRNA sequence by stochastically introducing one or more amino acid substitutions during protein translation,

(b) determining a first fraction of amino acid substitution at each potential amino acid position in the target protein sequence from the plurality of mistranslated variants,

(c) applying a functional selection criterion to the plurality of mistranslated variants in a functional assay,

(d) isolating a sub-set of mistranslated variants that conform to the functional criterion,

(e) determining a second fraction of amino acid substitution at each potential amino acid position in the target protein sequence from the sub-set of mistranslated variants isolated in step (d), and

(f) comparing the first fraction of amino acid substitutions to the second fraction of amino acid substitutions at each potential amino acid position in the target protein sequence.

In some embodiments, a lower second fraction of amino acid substitution compared to the first fraction of amino acid substitution for a position in the target protein indicates impaired functionality due to an amino acid substitution at the position in the target protein. In some embodiments, a higher second fraction of amino acid substitution compared to the first ratio of amino acid substitution for a position in the target protein indicates enhanced functionality due to an amino acid substitution at the position in the target protein.

In some embodiments of either aspect, each mistranslated variant independently comprises a substitution at less than about 40%, about 35%, about 30%, about 25%, about 20%, about 15%, about 10%, or about 5% of amino acid positions for each of one or more proteinogenic amino acid types in the target protein sequence. In some embodiments of either aspect, the mistranslated variants are generated in step (a) in a translation system comprising a living cell or a cell lysate. In further embodiments, the cell is a prokaryotic cell or eukaryotic cell. In further embodiments, stochastically introducing substitutions in step (a) comprises providing an amount of non-canonical amino acids in the translation system effective to compete with the corresponding canonical amino acids for mistranslation and incorporation into protein sequences at a desired frequency. In yet further embodiments, stochastically introducing substitutions further comprises providing an engineered amino acyl tRNA synthetase configured to increase frequency of mistranslation. In yet further embodiments, stochastically implementing substitutions in step (a) comprises providing the translation system with an amount of engineered tRNAs, or engineered aminoacyl tRNA synthetases, or a combination of an engineered tRNA and an engineered aminoacyl tRNA synthetase, that causes incorporation of a different amino acid residue than is canonically associated with a target codon. The different amino acid can be a different canonical amino acid. The engineered tRNAs or the engineered aminoacyl tRNA synthetases can be transgenically expressed by the cell.

In some embodiments of either aspect, the functional assay comprises detecting interaction of the plurality of mistranslated variants with a target molecule. In further embodiments, the target molecule is a small molecule, a nucleic acid, a peptide, or a protein. In further embodiments, the nucleic acid is DNA or RNA. In some embodiments, the target molecule is the target protein and the assay comprises detection of multimerization of the mistranslated variants. In some embodiments, the target molecule is an enzymatic substrate and the step of detecting interaction comprises detecting enzymatic activity. In some embodiments, the functional assay comprises detecting post-translational modifications in the plurality of mistranslated variants. In some embodiments of either aspect, the functional assay comprises a protein stability assay. In some embodiments of either aspect, the functional assay comprises a measurement of protein aggregation. In some embodiments of either aspect, determining the presence of amino acid substitutions comprises identification and quantification of peptides containing the amino acid substitution and peptides not containing the amino acid substitution by mass spectrometry.

In some embodiments of either aspect, the method further comprises performing the method separately for each of 2 or more proteinogenic amino acid types in the target protein sequence. In some embodiments, the method is performed separately for each of a plurality of amino acid types up to all 20 canonical amino acid types in the target protein sequence.

In some embodiments of either aspect, the method is performed simultaneously for a plurality of different target proteins. In further embodiments, the plurality of target proteins represents the proteome of a cell, or a substantial portion thereof.

In another aspect, the disclosure provides a method of screening amino acid substitutions in a target protein for enhanced functional characteristics, comprising performing the steps recited in above, and selecting one or more mistranslated variants that exhibit enhanced functionality compared to the target protein, and identifying the one or more substitutions in the one or more selected mistranslated variants associated with the enhanced functionality.

The innovative methods described herein presents an alternative to genetic-based analyses and provides a facile, high-throughput approach to rapidly generate amino acid substations representative of all or nearly all potential substitution sites for rapid testing and characterization. The methods described herein have an impact on detecting enhancing and deleterious substitutions, predicting sensitivity of domains to mutations, identification of pathogenic or disease related protein variant, and the like.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C schematically illustrate approaches to generate protein variants across the proteome by non-genetic mistranslation, whereby amino acid substitutions of a particular proteinogenic amino acid are introduced stochastically during protein translation. FIG. 1A illustrates mistranslation with non-canonical amino acids (ncAAs). FIG. 1B illustrates mistranslation with an engineered tRNA to create substitutions of the coding amino acid to a different proteinogenic amino acid. FIG. 1C represents a “statistical proteome” containing hundreds to thousands of protein variants for each expressed protein, as the result from the mistranslation approaches exemplified in FIGS. 1A and 1B.

FIGS. 2A and 2B schematically illustrate application of functional assays and related analytical framework to the statistical proteome illustrated in FIG. 1C. FIG. 2A illustrates performance of an exemplary embodiment of an enrichment functional assay. FIG. 2B illustrates performance of an exemplary embodiment of a profiling based assay, specifically a thermal denaturation assay.

FIGS. 3A-3E graphically illustrate experimental results of mistranslation using non-canonical amino acids (FIGS. 3A to 3D) or using engineered tRNAs (FIG. 3E). Specifically, FIGS. 3A and 3B graphically illustrate the levels of incorporation of non-canonical amino acids in cellular proteomes of Escherichia coli (FIG. 3A) and Saccharomyces cerevisiae (FIG. 3B), calculated as the percentage of peptides containing one or more non-canonical amino acids over the total peptides identified. FIG. 3C graphically illustrates a comparison of MS/MS spectra for a proline-containing peptide (bottom) and the same peptide where the ncAA L-azetidine-2-carboxylic acid (labeled as P1 in FIGS. 3A and 3B) replaces proline. These peptides were obtained from an S. cerevisiae lysate. FIG. 3D graphically illustrates the distribution of fractional incorporation values for L-azetidine-2-carboxylic acid on proline positions across the S. cerevisiae proteome. FIG. 3E graphically illustrates percent amino acid substitutions to a different proteinogenic amino acid (i.e., serine) at various codon sites using engineered mistranslating tRNAs.

FIGS. 4A-4C illustrate experimental results for enrichment-based affinity purification assays applied to protein variants generated by mistranslating the non-canonical amino acid azetidine 2-carboxylic acid (abbreviated as Aze in the figure), an analog of proline. In all panels, the functional selection criterion is affinity/interaction. The level of amino acid substitution on each proline position (Aze incorporation) is calculated as fractional amino acid substitution (signal of the peptide containing azetidine 2-carboxylic acid divided by the sum of intensities for both, the azetidine 2-carboxylic acid and the matched proline containing, peptides). FIG. 4A, top panel, schematically illustrates a target protein composed of three binding modules: a GST domain, an HA-tag, and a histidine-tag. FIG. 4A, bottom panel, illustrates effects of proline substitutions on the interactions of the different binding modules of the target protein (from top panel) to glutathione, an HA antibody, and cobalt ions, respectively. FIG. 4B graphically illustrates the effects of proline substitution on the assembly of the ribosome. FIG. 4C graphically illustrates the effects of proline substitutions on protein phosphorylation.

FIGS. 5A and 5B graphically illustrate experimental results for a thermal proteome profiling assay applied to mistranslated variants. Specifically illustrated are melting curves for matching proline (dashed) azetidine 2-carboxylic acid (Aze) (solid) containing peptides in UBC4 (FIG. 5A) and PGK1 (FIG. 5B).

### DETAILED DESCRIPTION

The present disclosure addresses a critical bottleneck in genome analysis and generally protein variant analysis, namely the determination of functional impacts of amino acid substitutions en masse. The disclosure is based on the inventors' development of an analytical platform initialy referred to as “limited mistranslation mutagenesis” (referred to hereinafter as “LMM”).

LMM is a technology that combines the generation of protein variants via non-genetic mistranslation with functional biochemical assays and mass spectrometry to assess the functional effects of amino acid substitutions on a proteome-wide basis within a relatively short timeframe. LMM generates unprecedented comprehensive collections of protein variants that can be analyzed in one-pot functional assays combined with mass spectrometry to generate sensitivity maps for a single protein or for the entire proteome, revealing deleterious, neutral, and./or advantageous amino acid substitutions. These maps will provide an invaluable resource for biologists, serving as an essential companion guide to genome sequences. Application of this technology can impact further studies of basic biology; protein engineering; and genomics.

In accordance with the foregoing, in one aspect, the disclosure provides a non-genetic, high-throughput method of introducing amino acid substitutions at a plurality of positions in a target protein. At a general level, mistranslation events are imposed stochastically during the translation of multiple copies of a target protein, resulting in a plurality of protein variants of the target protein. As a group, all of the variants of a particular target protein is referred to herein as an “ensemble” of protein variants or protein “quasi-species” of that reference target protein. The ensemble (or quasi-species), in its broadest sense, includes the reference sequence (i.e., wildtype or otherwise having no substitutions) in addition to all mistranslated variant species that have one or more substitutions imposed through the mistranslation event(s). In other instances, the plurality of protein variants is referred to as an ensemble of mistranslated protein variants. When performed simultaneously for all amino acid types for target proteins in a cell the aggregate of all the ensembles is referred to as a “statistical proteome”, which refers to the representation within the vast plurality of mistranslated proteins a statistical representation of all, or a substantial representation of all, the potential substitutions in the proteome.

In the methods disclosed herein, the ensemble of protein variants, or the statistical proteome, is subjected to functional assays and the observed results are compared to the reference or target protein (i.e., wildtype or otherwise having no substitutions).

Leveraging the power and sensitivity of mass spectrometry, protein fragments (i.e. peptides) containing amino acid substitutions can be identified and quantified, along with associated wild-type peptides. Relative changes upon the functional assay selection to the abundance of these peptides can be determined and associated with functional differences. Compiling such associations for all observed substitutions within the ensemble can permit generation of an aggregate map of the target or reference protein sequence with indications of the impact of substitutions at all potential (or practically observed) amino acid sites. Such a map can inform the functional role of each amino acid position in the overall protein and whether substitutions at that position result in a negative, positive, or neutral impact.

Generating the Ensemble of Protein Variants Using Limited Mistranslation

The methods disclosed herein comprise the step of generating a plurality of variants of a target protein (i.e., an ensemble of protein variants) from the same reference mRNA sequence by stochastically introducing one or more amino acid substitutions during protein translation. As indicated above, the plurality of variants of a target protein is translated from the same mRNA sequence. In this context, multiple mRNA molecules can be, and are typically, utilized in the translation system to produce the members of the plurality of variants. The phrase “same reference mRNA sequence” refers to the same encoding sequence that typically results in a polypeptide with the target amino acid sequence. Thus, in this context, the “same reference mRNA sequence” can include differences in the nucleic acid sequence that, by virtue of the redundancy of the genetic code, encode the same amino acid sequence under typical conditions (i.e., not mistranslation conditions).

The 20 naturally occurring amino acids that would typically occur in a wild-type target protein without any mistranslations are represented by the following abbreviations: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In some embodiments, the substitutions are of a single proteinogenic amino acid type in the implementation of this method step. For example, in performance of this step, substitutions of only prolines (Pro, P) are imposed. In another example, only arginines (Arg, R) are substituted. Any of the 20 proteinogenic amino acids can be the type that is targeted for potential substitution.

In alternative embodiments, the substitutions are imposed on more than a single proteinogenic amino acid type in the same pot reaction, such as 2, 3, 4, 5, 6, 7, 8, 9, 10, or more up to all 20 proteinogenic amino acid types can have substitutions imposed.

The substitutions are imposed stochastically by promoting limited mistranslation in the translation system, indicating that not all amino acids that are susceptible to substitution in the method are actually substituted. Instead, less than all potential amino acids are actually substituted. For example, in the context of the above embodiment where only one amino acid type (e.g., arginine) in the target protein is targeted for substitution, not all candidates (e.g., not all arginines) are actually substituted in the individual protein variants. Instead, there is a random or stochastic factor that governs whether any given amino acid (e.g., arginine) in the translated protein is actually substituted. The degree of substitution can be controlled or at least influenced to an extent by the altering concentrations and specificities of the mistranslation elements (e.g. non-canonical amino acids and engineered tRNAs) (as described in more detail below).

Regardless of the number of amino acid types that are targeted for substitution in the reaction pot, the total number of substitutions for any given resulting protein molecule produced is controlled to an extent. If an excessive number of substitutions are imposed in a single protein, the protein begins to lose its identifiable relationship with the wild-type or reference target protein. Furthermore, the presence of too many substitutions risks having multiple mutations affect each others' influence on the performance of the variant protein in a functional assay, effectively preventing assigning an observed functional effect to any individual substitution. Therefore, in some embodiments, a substantial proportion (e.g., about 70%, about 75%, about 80%, about 90%, about 95%, about 98%, about 99%, or all) of the members of the ensemble have less than 40% of their amino acid substituted. For example, the substantial proportion can have between about 1% to about 40%, about 2% to about 35%, about 3% to about 30%, about 4% to about 30%, about 5% to about 30%, about 5% to about 25%, about 2% to about 25%, about 2% to about 20%, about 1% to about 15%, about 5% to about 15%, and about 5% to about 10% of their amino acids substituted. In some embodiments, a substantial proportion of the mistranslated variants independently comprise a substitution at less than about 40%, about 35%, about 30%, about 25%, about 20%, about 15%, about 10%, or about 5% of amino acid positions for each of one or more proteinogenic amino acid types in the target protein sequence.

The mistranslated variants are generated in a translation system. The translation system can be any contained mixture of components required to translate a polypeptide molecule from an mRNA. Such a system comprises at least translation machinery including ribosomes, amino acyl-tRNA synthetases, and tRNAs. Exemplary translation systems include a living cell or a cell lysate or extract. The cells can be prokaryotic or eukaryotic. For example, as disclosed below, mistranslation was successfully conducted using both Escherichia coli and Saccharomyces cerevisiae.

The substitutions can be imposed stochastically using a variety of non-genetic strategies. The term “non-genetic” refers to incorporation of steps to impose substitutions in polypeptide molecules that do not rely on mutating the encoding DNA or mRNA transcript.

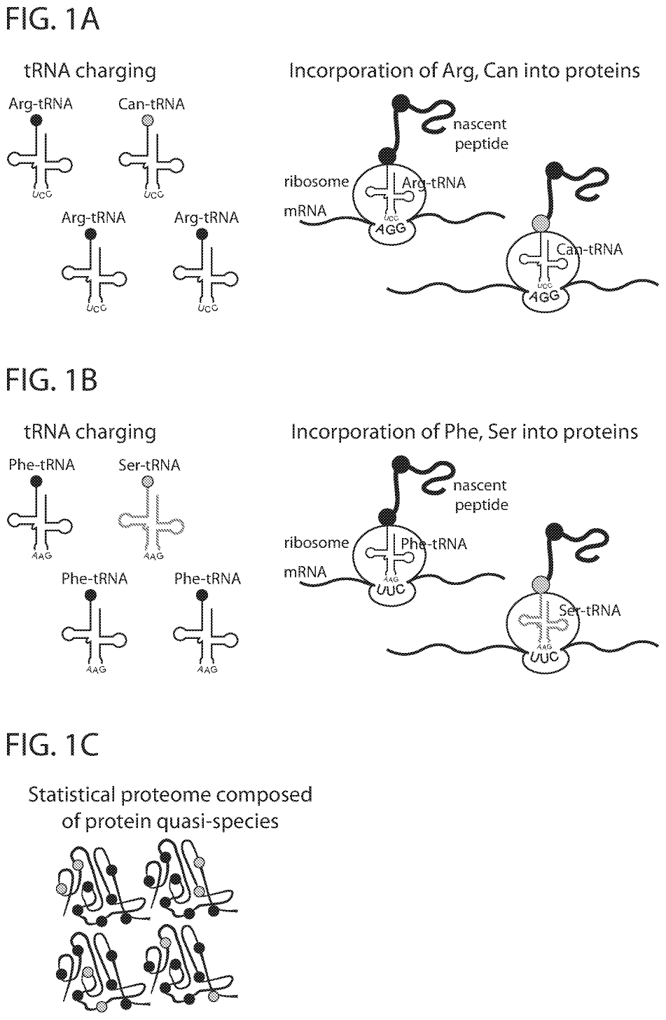

In one embodiment, stochastically introducing substitutions comprises providing the translation system with an amount of a non-canonical amino acid (ncAA). In some embodiments, the translation system is provided with amounts of two or more different ncAAs to promote mistranslation. tRNAs and the tRNA synthetases are somewhat promiscuous and will occasionally permit charging of a tRNA with an ncAA that exhibits some structural similarity to the cognate amino acid. Thus, when present in sufficient concentration, ncAAs will be charged onto tRNA and will then be integrated into a translated protein during the translation process, resulting in a mistranslation event. A schematic representation of this approach is provided in FIG. 1A. As illustrated, an exemplary translation system is supplied with the ncAA canavanine (Can). Canavanine is sufficiently similar to natural arginine (Arg) to be recognized by the arginyl-tRNA synthetase that typically charge the Arg onto the arginine tRNAs. Thus, when recognized, arginyl-tRNA synthetase will stochastically charge Can onto the arginine tRNAs instead of Arg and ultimately incorporate the Can into proteins in place of their cognate Arg during protein synthesis. In the figure, we show the ncAA, which can be recognized by the arginyl-tRNA synthetase, charged onto tRNA in place of arginine (Arg) and stochastically incorporated into arginine (Arg) positions.

By controlling the concentration of the one or more ncAAs, the rate of tRNA charging with the ncAAs can be influenced, which then influences the rate of substitution in each (mis)translated protein variant. A person of ordinary skill in the art can readily optimize the imposition of a desired rate of substitution by appropriately controlling the concentration of ncAA's in the translation system

Exemplary ncAAs and their canonical counterparts (represented with single letter abbreviations) include:

A: 2-aminoisobutyric acid; A: 1-amino-1-cyclopropanecarboxylic acid; D: DL-threo-β-methylaspartic acid; E: 4-fluoro-glutamic acid; F: 4-amino-L-phenylalanine; F: 4-fluoro-phenylalanine; F: 2-amino-3-phenylbutanoic acid; I: cyclohexylglycine; K: 4-thialysine; L: 4-aza-leucine; L: L-β-tertbutylalanine; L: cyclopentylalanine; M: ethionine; M: norleucine; M: L-γ-azidohomoalanine; P: L-azetidine-2-carboxylic acid; P: thiazolidine-2-carboxylic acid; P: L-thiazolidine-4-carboxylic acid; P: trans-4-hydroxy-L-proline; Q: L-theanine; R: canavanine; R: L-homoarginine; V: tert-leucine; V: (S)-2-amino-2-cyclobutylacetic acid (also known as L-cyclobutylglycine); W: 5-fluoro-tryptophan; W: L-5-hydroxytryptophan; W: H-β-(3-benzothienyl)-alanine; Y: 3-fluoro-tyrosine; Y: 3-nitro-L-tyrosine. See also, for example, Williams et al., Mol. Cell. Biol. 9:2574 (1989); Evans et al., J. Amer. Chem. Soc. 112:4011-4030 (1990); Pu et al., J. Amer. Chem. Soc. 56:1280-1283 (1991); Williams et al., J. Amer. Chem. Soc. 113:9276-9286 (1991); and Budisa, N.. Engineering the Genetic Code: Expanding the Amino Acid Repertoire for the Design of Novel Proteins, Wiley-VCH Verlag GmbH & Co. KGaA, 2006; and all references cited therein, each of which is incorporated herein by reference in its entirety.

In other embodiments, stochastic introduction of a substitution can be implemented by providing altered tRNA or amino acyl tRNA synthetase in the translation system to compete with the typical tRNA or amino acyl tRNA synthetase. These embodiments are not limited to substituting ncAAs but rather allow for the substitution of different proteinogenic amino acids, which can be rationally imposed.

For example, in a specific embodiment, introducing substitutions comprises providing the translation system with an amount of one or more altered or engineered tRNA. A wild-type tRNA can be altered at its anticodon, thus changing the specificity of the tRNA for a different codon. The result is that the amino acid charged on the tRNA will be imposed at an alternate target codon. The engineered tRNA with the modified codon will compete with the naturally occurring tRNA of the same codon (charged with the cognate amino acid for that codon), resulting in a stochastic factor for a substitution. Codons for all canonical amino acids are known. Therefore, the design of any altered or engineered tRNA can be rationally implemented to impose a desired substitution. FIG. 1B schematically illustrates imposition of an exemplary mistranslation event using altered tRNA to result in substitutions with alternative proteinogenic amino acids. In the example shown, a mutant serine-tRNA has an anticodon that recognizes a phenylalanine codon. As a result, serine is incorporated stochastically into phenylalanine positions. The result is an ensemble of proteins that have phenylalanine to serine substitutions.

In other embodiments, modified or engineered amino acyl tRNA synthetases can be introduced into the translation system. This results in charging of alternative proteinogenic amino acids on tRNAs. The result is the same in that a different amino acid is incorporated in a polypeptide with some frequency at a codon typically associated with different cognate amino acid.

The engineered tRNAs or amino acyl tRNA synthetases can be physically added to the translation system or, in cases of live cells, can be transgenically expressed in the cell itself at a rate that competes with the endogenous tRNAs or amino acyl tRNA synthetases at a desired level.

The above approaches for stochastically inducing substitutions via mistranslation can be combined. For example, the translation system can be provided with a mixture of ncAAs, engineered tRNAs, and/or engineered amino acyl tRNA synthetase.

There is no limit to the number of individuals in the ensemble of mistranslated protein variants. The limit is simply imposed by the practical limitations of the translation system. As indicated above, the rate of substitution is controlled to avoid excessive alterations in the variants. Thus, a sufficient number of variants is desired such that even with a relatively low substitution rate (e.g., about 5% of amino acids in a given variant), the aggregate of the ensemble will contain a representative of substantially all of the potential substitutions in the target protein sequence.

In embodiments, where only a single proteinogenic amino acid type (or limited number thereof) is targeted for substitution, the method can be performed in parallel for other proteinogenic amino acid types. For example, 20 different ensembles can be produced in parallel for the same target protein, wherein each ensemble has variants of a single type of substitution (e.g., at proline residues).

The above discussion generally addresses the method in the context of generating an ensemble of mistranslated protein variants of a single target protein. However, the method can address multiple target proteins simultaneously in a single pot reaction. The imposition of mistranslation can result in a “statistical proteome”, as represented by FIG. 1C, where the aggregate of individual variants (with only relatively few substitutions per variant) combines to represent a substitution at every (or nearly every) potential position in the proteome.

Functional Assays

In some embodiments, the method further comprises subjecting an ensemble (i.e., plurality of mistranslated variants) of a target protein to a functional assay. The parameters imposed in the assay can facilitate determination of the role or impact of the observed substitutions on the target protein.

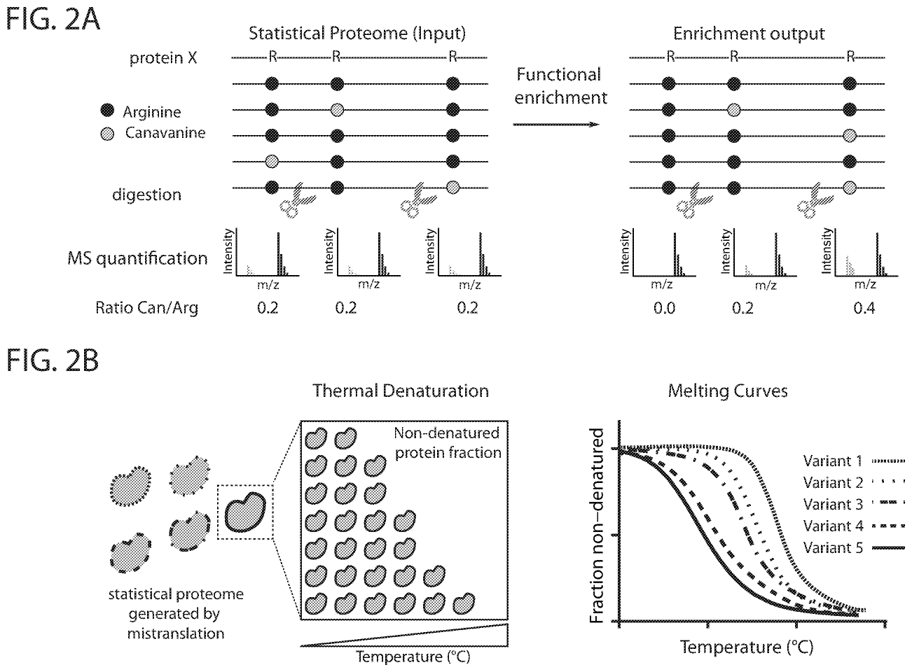

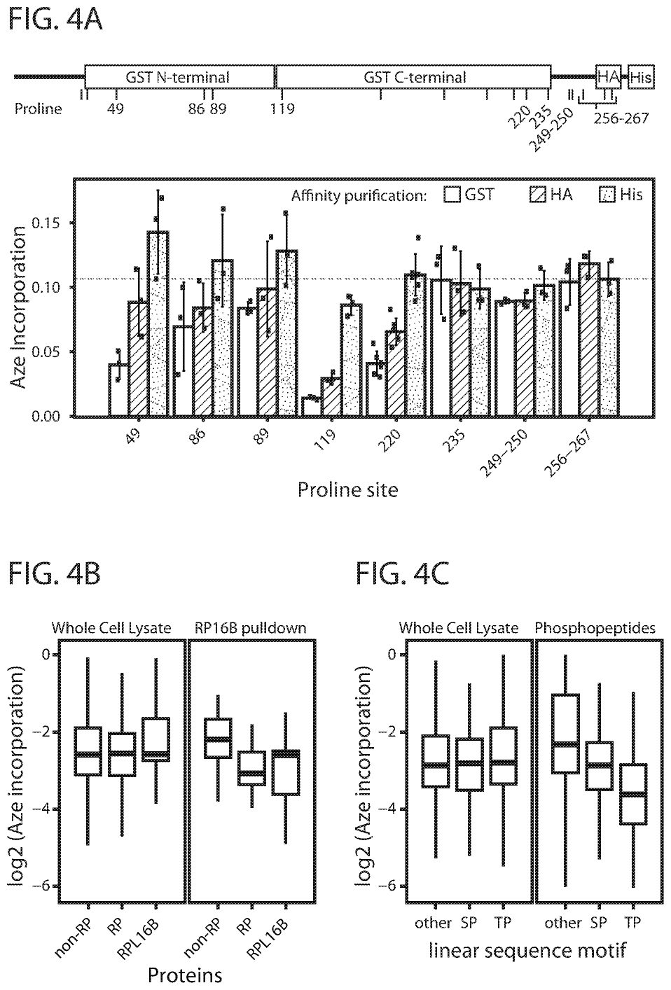

One embodiment of a functional assay is an enrichment assay, which is schematically illustrated in FIG. 2A. In an enrichment assay, the ensemble of mistranslated variants (potentially including the wild-type or reference target protein), or even a statistical proteome, is subjected to a biochemical assay that separates variants by a function or physical property. Enrichment-based assays typically impose a functional selection to the ensemble of mistranslated variants (or statistical proteome). The mistranslated variants (or statistical proteome) are analyzed before and after the selection. In some embodiments, the analysis comprises proteolysis of a sample of the proteins by enzymatic digestion. The resulting peptides are analyzed by mass spectrometry to identify a sequence and quantify their abundance. Peptide pairs are matched and a mass spectrometry signal ratio between the peptide containing the ncAA (in FIG. 2A, canavanine, or Can) and the cognate amino acid (in FIG. 2A, arginine, or Arg) is calculated. This ratio can also be calculated as fractional amino acid substitution (i.e., signal of the peptide containing ncAA divided by the sum of intensities for both peptides). Positions, such as the first arginine position shown in the protein in the FIG. 2A, where the Arg-to-Can substitution has a detrimental effect on function will show a decrease in the Can/Arg ratio after selection with respect to the sample prior to enrichment and with respect to other positions. This analysis is conducted for all the arginine positions in the ensemble (or proteome) that are measured. Specific examples of this type of assay are discussed in more detail below and illustrated in FIGS. 4A-4C. As indicated above, this assay can be applied to ensembles (or the statistical proteome) generated in parallel to impose stochastic substitutions of other proteinogenic amino acid type(s).

Thus, in some embodiments, the method further comprises the following steps:

determining a first fraction of amino acid substitution at each potential amino acid position in the target protein sequence from the plurality of mistranslated variants,

applying a functional selection criterion to the plurality of mistranslated variants in a functional assay,

isolating a sub-set of mistranslated variants that conform to the functional criterion, and

determining a second fraction of amino acid substitution at each potential amino acid position in the target protein sequence from the sub-set of isolated mistranslated variants.

In some embodiments, the selection method further comprises comparing the first fraction of amino acid substitutions to the second fraction of amino acid substitutions at each potential amino acid position in the target protein sequence. In some embodiments, a lower second fraction of amino acid substitution compared to the first fraction of amino acid substitution for a position in the target protein indicates impaired functionality due to an amino acid substitution at the position in the target protein. In other embodiments, a higher second fraction of amino acid substitution compared to the first ratio of amino acid substitution for a position in the target protein indicates enhanced functionality due to an amino acid substitution at the position in the target protein.

Non-limiting examples of such selection-type functional assays that impose a selection criterion can include assays that detect interaction with a target molecule. Exemplary target molecules include small molecules, nucleic acids (e.g., DNA or RNA), other proteins, and the like. For example, a target molecule can be attached to a substrate and interaction can be determined by the physical isolation of protein variants that successfully interact with the target molecule. In some embodiments, the assays detect multimerization of the variants with each other or with a non-mutated version of the target protein itself. In other embodiments, the target molecule is an enzyme and detecting interaction comprises detecting enzymatic activity.

Another example of an enrichment assay is to test protein degradation. To illustrate, mistranslated statistical proteomes in yeast can be generated in the presence or absence of proteasome inhibitors and amino acid substitution rates quantified at every relevant position in both conditions. Comparison of amino acid substitution rates between untreated and proteasome inhibitor treated samples enable identification of positions that, upon amino acid substitution, lead to changes in proteasomal degradation.

In other embodiments, the functional assay is a profiling assay. Profiling-based assays impose a set of defined conditions to the ensemble of mistranslated variants (or even a statistical proteome). Such defined conditions can be, for example, a range of pH, a range of concentrations of a chemical, a concentration of a salt, a panel of different buffers, or a range of temperatures. Fitness across conditions for mistranslated variants is assessed by mass spectrometry and profile curves are derived for wild type peptides and matched peptides containing amino acid substitution. FIG. 2B schematically represents an illustrative example of a thermal denaturation assay performed on a mistranslated statistical proteome. Proteins that aggregate are removed from solution and the non-denatured protein fraction is analyzed by mass spectrometry to identify and quantify peptides across the various temperature conditions. Melting curves are extracted for variants and compared to wild type to assess effect of amino acid substitutions on protein stability.

In another illustrative example, a profiling-based assay as described in FIG. 2B could be implemented to identify amino acid substitutions that alter protein subcellular localization. Cells resulting from either ncAA or mutant tRNA mistranslation can be homogenized in isotonic conditions to preserve organellar organization and then subjected to subcellular fractionation by ultracentrifugation using a sucrose gradient. In such an approach, an abundance profile across cellular compartments would be obtained for mistranslated protein variants, and the impact of each amino acid substitution assessed by comparison to variants that have the canonical amino acid on that position. Similarly, statistical proteomes can be subjected to size exclusion chromatography fractionation and abundance profiles obtained for protein variants in order to understand the effects of amino acid substitutions on the multimerization states of proteins.

Accordingly, in some embodiments, the method further comprises the steps of

applying the plurality of mistranslated variants to defined conditions in functional assays,

measuring a value for the variants containing each amino acid substitution and a value for the variants that do not contain that amino acid substitution, and

comparing the value for the variants containing each amino acid substitution and a value for the variants that do not contain that amino acid substitution.

In some embodiments, the method yet further comprises associating differences in the values with amino acid positions that are important for the structure or function of the target protein.

In some embodiments, the defined conditions comprise a range of temperatures, pH values, chemical concentrations, or salt concentrations, and measuring a value comprises measuring the solubility profile of the plurality of mistranslation variants across the range.

Additional examples of profiling assays include assaying subcellular localization or degradation of the mistranslation variants.

In any of the above embodiments, e.g., whether utilizing a functional enrichment assay or a profiling assay, the method can incorporate the active step of determining the presence of the amino acid substitution, e.g., by identification and quantification of peptides containing the amino acid substitution and peptides not containing the amino acid substitution by mass spectrometry. The identification and/or detection of a substitution can be correlated to the effect in the functional assay. The method can further comprise compiling values associated with each substitution site reflective of the effect on the target protein into a map of the target protein sequence. The type of value and the functional inference will depend on the type of assay, as described above.

The method described herein can be integrated into a screen for amino acid substitutions in a target protein in support of protein engineering design. The method described above can be performed to detect and identify substitutions that enhance the functionality of a target protein under defined conditions (e.g., the ability to bind a target molecule, to enzymatically catalyze a reaction, to avoid degradation, and the like). Once identified, the substitution can be incorporated into the protein and transgenically expressed. In other applications, the compiled map of a target protein can be informative to identify a region of the target protein that is sensitive to mutation, i.e., that requires maintenance of the wild-type sequence for functionality. This region could be preserved while directing modifications to other regions of the protein.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); Coligan, J. E., et al. (eds.), Current Protocols in Immunology, John Wiley & Sons, New York (2010); Mirzaei, H. and Carrasco, M. (eds.), Modern Proteomics—Sample Preparation, Analysis and Practical Applications in Advances in Experimental Medicine and Biology, Springer International Publishing, 2016; and Comai, L, et al., (eds.), Proteomic: Methods and Protocols in Methods in Molecular Biology, Springer International Publishing, 2017, for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following discussions are set forth so as to provide those of ordinary skill in the art with a complete disclosure and description of how to make and use the disclosed innovations, and are not intended to limit the scope of what the inventors regard as their invention nor are they intended to represent that the experiments below are all or the only experiments performed.

This first discussion presents exemplary strategies to generate protein variants via mistranslation and identify these variants by mass spectrometry. These approaches can facilitate rapid and broad functional assessment of amino acid substitutions across the proteome while avoiding the need for genetic manipulations of the associated protein-coding genes.

Suites or ensembles of mistranslated protein variants were produced by two methods. In the first method, different non-canonical amino acids were added to the growth media of Escherichia coli and Saccharomyces cerevisiae in individual parallel assays (i.e., one non-canonical amino acid type added per assay). The suite of non-canonical amino acids applied were (abbreviations were defined so the letter corresponds to the cognate natural amino acid and the number is an arbitrary number for cross reference with data presentation): A1: 2-aminoisobutyric acid; A2: 1-amino-1-cyclopropanecarboxylic acid; D1: DL-threo-β-methylaspartic acid; E1: 4-fluoro-glutamic acid; F1: 4-Amino-L-phenylalanine; F2: 4-fluoro-phenylalanine; F3: 2-amino-3-phenylbutanoic acid; I1: cyclohexylglycine; K1: 4-Thialysine; L1: 4-aza-leucine; L2: L-β-tertbutylalanine; L3: cyclopentylalanine; M1: ethionine; M2: norleucine; M3: L-γ-azidohomoalanine; P1: L-azetidine-2-carboxylic acid; P2: thiazolidine-2-carboxylic acid; P3: L-thiazolidine-4-carboxylic acid; P4: trans-4-hydroxy-L-proline; Q1: L-theanine; R1: canavanine; R2: L-homoarginine; V2: tert-leucine; V3: (S)-2-amino-2-cyclobutylacetic acid (also known as L-cyclobutylglycine); W1: 5-fluoro-tryptophan; W2: L-5-hydroxytryptophan; W3: H-β-(3-benzothienyl)-alanine; Y1: 3-fluoro-tyrosine; Y2: 3-nitro-L-tyrosine.

To illustrate the method, yeast strain BY4742 was grown in lysine drop out synthetic complete media at 30° C. to optical density OD₆₀₀ of 0.2, at which point non-canonical amino acid were added. In two exemplary assays, arginine analog canavanine was added to the growth media at 75 μg/ml and Proline analog azetidine-2-carboxylic acid was added to its culture at 90 μg/ml.

The naturally occurring amino acyl-tRNA synthetases have evolved to distinguish the twenty proteinogenic amino acids. They are however promiscuous towards non-canonical amino acids that resemble their cognate substrates. Thus, provided they are present at sufficient concentrations, certain non-canonical amino acids will be charged onto cognate tRNAs and subsequently incorporated into proteins (see, e.g., FIG. 1A). Standard cell cultures were maintained to facilitate translation and, thus, production of a statistical proteome of protein mistranslated variants (FIG. 1C). For example, the yeast-based cultures were harvested at optical density OD₆₀₀ 1. Yeast cell pellets were harvested by centrifugation at 2,850 g at 4° C. Cell pellets were resuspended in ice-cold sterile water and pelleted again at 10,600 g at 4° C. Cell pellets were snap-frozen in liquid nitrogen and stored at −80° C. until cell lysis. The statistical proteomes produced in the cell cultures were analyzed using mass spectrometry. Briefly, harvested E. coli or S. cerevisiae cells were resuspended in lysis buffer containing phosphatase and protease inhibitors. Cell lysis was carried out by either bead beating or sonication and protein lysates were reduced and alkylated prior to enzymatic digestion. Proteins were digested overnight with either trypsin or lysyl-endopeptidase, followed by acidification and peptide desalting. Desalted peptides were resuspended in 5% acetonitrile and 5% formic acid and subjected to liquid chromatography coupled to tandem mass spectrometry (LC-MS/MS). Online analysis of peptides was carried out on a hybrid ion trap orbitrap (LTQ Orbitrap Velos) or a hybrid quadrupole orbitrap (Q-Exactive) mass spectrometer (Thermo Fisher Scientific) using data-dependent acquisition methods. MS data was searched against the S. cerevisiae or E. coli protein sequence databases using the Comet search engine, and setting as variable modifications the mass differences corresponding to the expected amino acid substitutions. Results were filtered with Percolator to 1% false discovery rate at the level of peptide spectral matches.

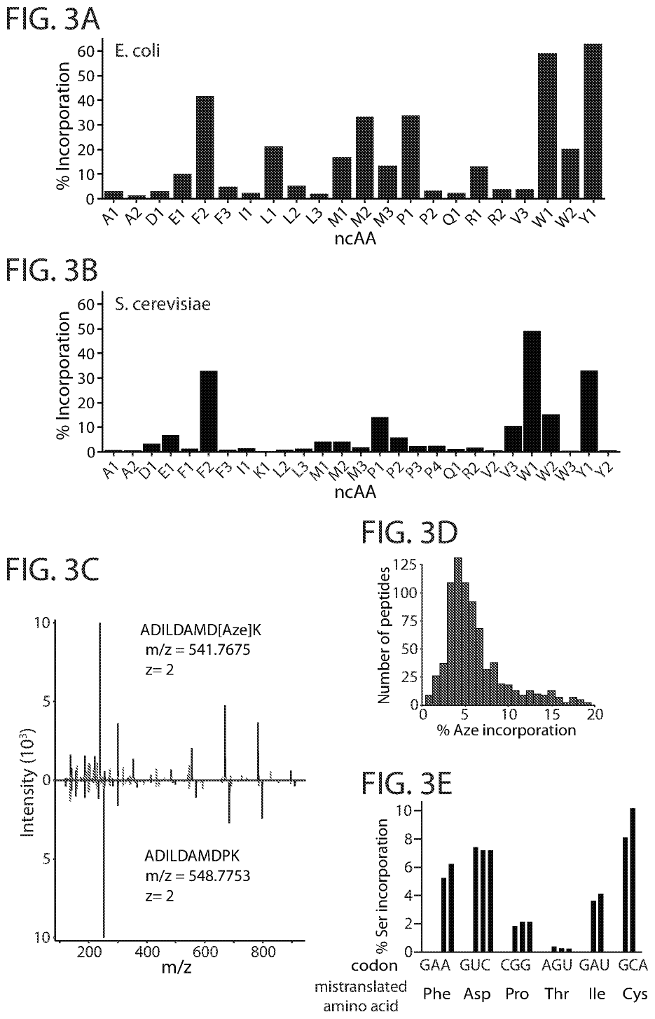

FIGS. 3A-3D illustrate the results of the forced mistranslation utilizing the suite of non-canonical amino acids. FIGS. 3A and 3B show the observed incorporation of non-canonical amino acids in the cellular proteomes. Specifically, percentages of unique peptides showing amino acid misincorporation of the various non-canonical amino acids at cognate amino acid sites are represented for Escherichia coli (FIG. 3A) and Saccharomyces cerevisiae (FIG. 3B). Ranges of incorporation varied widely, reaching close to 60% for some amino acids. This illustrates that use of non-canonical amino acids can be used in cellular systems to impose stochastic substitutions throughout the proteome without requiring the tedious steps of genetic manipulation.

FIG. 3C illustrates an exemplary comparison of MS/MS spectra for a proline-containing peptide (bottom) and the same peptide where the non-canonical amino acid L-azetidine-2-carboxylic acid (P1 from FIG. 3A and 3B) replaces proline obtained from a S. cerevisiae lysate. Both spectra show similar intensities for most peptide matched fragment ions (solid black lines). In the figure, L-azetidine-2-carboxylic acid is represented with the “Aze” abbreviation and “m/z” represents mass over charge. FIG. 3D illustrates the distribution of fractional incorporation values for the non-canonical amino acid L-azetidine-2-carboxylic acid (Aze) on proline positions across the proteome. For this particular assay, the Saccharomyces cerevisiae was grown in media containing the non-canonical amino acid analog azetidine-2-carboxylic. Cellular lysates were prepared and peptides identified and quantified by mass spectrometry. Fractional incorporation was calculated as the intensity of the peptide containing L-azetidine-2-carboxylic acid divided by the summed intensity of the peptide pair (peptide with L-azetidine-2-carboxylic acid and wild-type peptide), and indicated as a percentage. In this experiment, the majority of the proline positions are substituted by L-azetidine-2-carboxylic acid at about 5%.

These data illustrate that amino acids of a target protein can be stochastically substituted at an appropriate rate to allow for detectable variants for purposes of functional assays that can be compared against the wild type or reference sequence.

Moreover, the substitutions can be identified by mass spectrometry, thus obviating the need for serial modification of the DNA to introduce and identify mutations. When applied to all amino acid types and at a sufficiently large scale (i.e., in a cell), this non-genetic approach of stochastically imposing mutations for all proteinogenic amino acid types can provide sufficient mutational coverage to comprehensively assay the amino acid positions in the proteome.

In the second method, engineered tRNA's were introduced into S. cerevisiae cells via transgenic expression. The effect of the engineering is to allow the tRNA specific for a particular amino acid to have a different codon, thus imposing the incorporation of the amino acid residue at a codon not typically associated with that residue. The rate of this misincorporation is influenced by the concentration of the engineered tRNA compared to the wild-type version with the canonical (unmutated) codon. To generate the data illustrated in FIG. 3E, serine tRNAs engineered with altered codons were expressed in the S. cerevisiae cells. Specifically, expression of Ser tRNAs with anticodon mutations caused mistranslation at the targeted codon sites, creating a mixture of protein variants with serine substitutions. The targeted codons were GAA (Phe), GUC (Asp), CGG (Pro), AGU (Thr), GAU (Ile) and GCA (Cys). FIG. 3E specifically illustrates the observed serine substitution rates at the various target codons as a result of the presence of the engineered mistranslating tRNAs. Peptides containing serine substitutions were identified and quantified by mass spectrometry, as described above. In the figure, the percentage of peptides containing the target amino acid for which wild type and serine-substituted versions were detected is plotted.

These data demonstrate that mistranslation can be readily imposed by simply modifying a region of the tRNA sequence in the protein translation system (e.g., cell) to have altered anticodons. tRNAs for any of the canonical amino acids can be readily modified to alter the target anticodon, thereby permitting targeted substitutions of one amino acid for another amino acid type. Depending on the relative proportion of a tRNA that has the mutated anticodon, the desired amino acid substitution can be imposed at a desired rate.

This second discussion presents exemplary embodiments of applying the target proteins and the ensembles of corresponding mistranslated variants (i.e., the statistical proteome) generated by mistranslation to functional assays. As a result, the spectrum of protein substitutions is functionally assessed for impacts on the corresponding target proteins.

In an enrichment approach, the statistical proteome, generated as described above, is applied to an enrichment-based functional assay. An example of the approach is schematically represented in FIG. 2A. With reference to a single protein (although the entire proteome can be assessed en masse), the target protein and its mistranslated variants are analyzed by mass spectrometry before and after a selection criterion is applied. For example, the proteins are proteolyzed enzymatically and peptides are analyzed by mass spectrometry to identify a sequence and quantify its abundance. Peptide pairs are matched and a mass spectrometry signal ratio between the peptide containing the substituted (e.g., non-canonical) amino acid and the cognate amino acid (e.g., the wild-type) is calculated.

An enrichment-based affinity purification assay was applied to protein variants generated by mistranslating the non-canonical amino acid azetidine 2-carboxylic acid (referred to as “Aze”) in place of its proteinogenic analog proline. A functional selection criteria of affinity/interaction was applied and the amino acid substitution ratio as fractional amino acid substitution (signal of the peptide containing azetidine 2-carboxylic acid divided by the sum of intensities for both peptides) was calculated. Specifically, FIG. 4A addresses the application of the mistranslation approach to a protein composed of three binding modules: a GST domain, an HA-tag, and a histidine-tag (see diagram in the top panel). The protein was expressed using an in vitro translation system derived from HeLa cells, in the presence of the proline analog azetidine 2-carboxylic acid (Aze). Then, a denaturing purification was conducted against the histidine tag to assess Aze incorporation at proline positions. Furthermore, native affinity purifications against GST and HA-tag were performed to assess the impact of proline-to-Aze replacements on protein structure and interactions. Using mass spectrometry, the relative abundance was measured between the Aze-containing peptides and their corresponding wild-type forms for 8 out of the 17 prolines in the protein (see bottom panel). Proline positions in the protein sequence are indicated in the x-axis. As illustrated, there was approximately 10% incorporation of Aze across proline positions in the denaturing histidine-tag purification. In contrast, multiple positions were significantly depleted of Aze in both native purifications (1-4% Aze). The two purifications shared the same effects, yet the magnitude was greater on the GST purification, in particular for two residues near the start of the N-terminal GST and C-terminal GST modules. This suggests that most substitutions have an effect on protein folding while a few specific substitutions also disrupt interactions.

The effects of proline to Aze substitutions on the assembly of the ribosome were then studied. Azetidine 2-carboxylic acid (Aze) was introduced into S. cerevisiae strain expressing a ZZ-tagged ribosomal protein subunit (RPL16B), according to the approach described above. Whole cellular lysate was prepared and ribosomes were purified using protein IgG affinity beads. The ratio of incorporation of Aze at proline positions (expressed as log2) was determined for purified ribosomes (FIG. 4B, right panel) and whole cell native lysate (FIG. 4B, left panel). Peptides from ribosomal (RP), non-ribosomal proteins (non-RP) and the tagged protein RPL16B are represented on different series. As illustrated in FIG. 4B, azetidine is generally depleted in ribosomal proteins on the RPL16B pull-down, indicating that generally proline substitutions disrupt ribosome assembly.

The effects of proline-to-Aze substitutions on protein phosphorylation were also assayed. Azetidine 2-carboxylic acid (Aze) was introduced into S. cerevisiae and the ratio of incorporation of Aze at proline positions (expressed as log2) was determined for non-phosphorylated peptides (FIG. 4C, left panel), and phosphopeptides (FIG. 4C, right panel). As shown, Aze is depleted on phosphopeptides containing an SP (serine followed by proline) or TP (threonine followed by proline) motif, showing the negative effect of proline substitutions on phosphorylation of the adjacent phosphoacceptor residue.

In a profiling-based assay, the statistical proteome, generated as described above, is subjected to defined assay conditions and a performance (e.g., protein fitness) profile by members of the statistical proteome under the conditions is generated. An example of the approach is schematically represented in FIG. 2B. As illustrated, a set of defined conditions are applied to the statistical proteome, for example a range of pH, a range of concentrations of a chemical, a concentration of a salt, a panel of different buffers, or a range of temperatures. Fitness across conditions for mistranslated variants is assessed by mass spectrometry and profile curves are derived for wild-type peptides and matched peptides containing amino acid substitution(s).

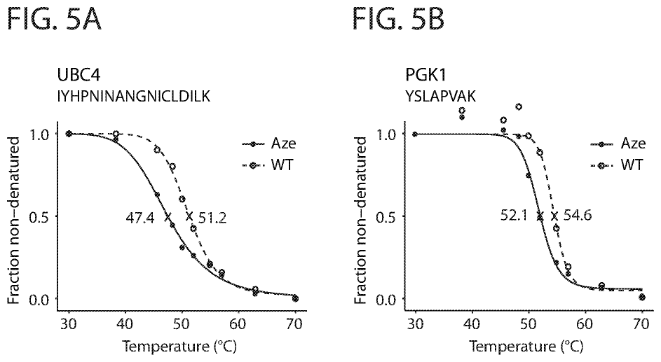

FIG. 5 provides results for an exemplary profiling assay performed on a statistical proteome. Specifically, the experimental results are illustrated for a thermal proteome profiling assay applied to mistranslated variants. Following azetidine-2-carboxylic acid (Aze) misincorporation in S. cerevisiae, native protein lysates were prepared. Thermal denaturation was carried out as explained in FIG. 2B, followed by quantification of the soluble proteins at each temperature point by mass spectrometry. Melting curves represent stability for protein variants and melting point (Tm) is determined at the inflection point where 50% of the protein remains in the soluble fraction. Melting curves for matching proline (dashed) and Aze (solid) containing peptides in UBC4 (FIG. 5A) and PGK1 (FIG. 5B) proteins show that Aze substitutions at these sites have a deleterious impact on protein stability.

These data demonstrate that functional assays can be applied to statistical proteomes generated using the mistranslation approach described herein to provide informative data regarding the functional impact of a substitution of a wild-type amino acid in a target protein. Mistranslation can be performed for each proteinogenic amino acid type. Due to the sensitivity, all proteins in a cell can be assessed simultaneously and functional maps of protein substitutions can be rapidly generated.

While illustrative embodiments have been illustrated and described, it will be appreciated that various changes can be made therein without departing from the spirit and scope of the invention.

The embodiments of the invention in which an exclusive property or privilege is claimed are defined as follows:

1. A non-genetic, high-throughput method of introducing and assaying effects of amino acid substitutions at a plurality of positions in a target protein, comprising:
(a) generating a plurality of variants of a target protein from the same reference mRNA sequence by stochastically introducing one or more amino acid substitutions during protein translation,
(b) applying the plurality of mistranslated variants to defined conditions in functional assays,
(c) measuring a value for each of the variants containing each amino acid substitution and a value for a variant that does not contain that amino acid substitution,
(d) comparing the values for the variants containing each amino acid substitution and the value for the variant that does not contain that amino acid substitution, and
(e) associating differences in the values with amino acid positions that are important for the structure or function of the target protein.

2. The method of claim 1, further comprising determining the presence of a substitution at one or more positions in each of the plurality of mistranslated variants.

3. The method of claim 1, further comprising compiling the differences in values determined in step (d) into a functional map of the target protein sequence.

4. The method of claim 1, wherein the defined conditions in step (b) comprise a range of temperatures, pH values, chemical concentrations, or salt concentrations, and measuring a value comprises measuring the solubility profile of the plurality of mistranslation variants across the range.

5. The method of claim 1, wherein the functional assay of step (b) comprises assaying subcellular localization.

6. The method of claim 1, wherein the functional assay comprises assaying degradation of the plurality of mistranslated variants.

7. A non-genetic, high-throughput method of assaying effects of amino acid substitutions at a plurality of positions in a target protein, comprising:
(a) generating a plurality of variants of a target protein from the same reference mRNA sequence by stochastically introducing one or more amino acid substitutions during protein translation,
(b) determining a first fraction of amino acid substitution at each potential amino acid position in the target protein sequence from the plurality of mistranslated variants,
(c) applying a functional selection criterion to the plurality of mistranslated variants in a functional assay,
(d) isolating a sub-set of mistranslated variants that conform to the functional criterion,
(e) determining a second fraction of amino acid substitution at each potential amino acid position in the target protein sequence from the sub-set of mistranslated variants isolated in step (d), and
(f) comparing the first fraction of amino acid substitutions to the second fraction of amino acid substitutions at each potential amino acid position in the target protein sequence.

8. The method of claim 7, wherein a lower second fraction of amino acid substitution compared to the first fraction of amino acid substitution for a position in the target protein indicates impaired functionality due to an amino acid substitution at the position in the target protein.

9. The method of claim 7, wherein a higher second fraction of amino acid substitution compared to the first ratio of amino acid substitution for a position in the target protein indicates enhanced functionality due to an amino acid substitution at the position in the target protein.

10. The method of any one of claims 1-9, wherein each mistranslated variant independently comprises a substitution at less than about 40%, about 35%, about 30%, about 25%, about 20%, about 15%, about 10%, or about 5% of amino acid positions for each of one or more proteinogenic amino acid types in the target protein sequence.

11. The method of any one of claims 1-9, wherein the mistranslated variants are generated in step (a) in a translation system comprising a living cell or a cell lysate.

12. The method of claim 11, wherein the cell is a prokaryotic cell or eukaryotic cell.

13. The method of claim 11, wherein stochastically introducing substitutions in step (a) comprises providing an amount of non-canonical amino acids in the translation system effective to compete with the corresponding canonical amino acids for mistranslation and incorporation into protein sequences at a desired frequency.

14. The method of claim 13, where stochastically introducing substitutions further comprises providing an engineered amino acyl tRNA synthetase configured to increase frequency of mistranslation.

15. The method of claim 11, wherein stochastically implementing substitutions in step (a) comprises providing the translation system with an amount of engineered tRNAs, or engineered aminoacyl tRNA synthetases, or a combination of an engineered tRNA and an engineered aminoacyl tRNA synthetase, that causes incorporation of a different amino acid residue than is canonically associated with a target codon.

16. The method of claim 15, wherein the different amino acid is a different canonical amino acid.

17. The method of claim 15, wherein the engineered tRNAs or the engineered aminoacyl tRNA synthetases are transgenically expressed by the cell.

18. The method of claim 1 or claim 7, wherein the functional assay comprises detecting interaction of the plurality of mistranslated variants with a target molecule.

19. The method of claim 18, wherein the target molecule is a small molecule, a nucleic acid, a peptide, or a protein.

20. The method of claim 19, wherein the nucleic acid is DNA or RNA.

21. The method of claim 19, wherein the target molecule is the target protein and the assay comprises detection of multimerization of the mistranslated variants.

22. The method of claim 18, wherein the target molecule is an enzymatic substrate and the step of detecting interaction comprises detecting enzymatic activity.

23. The method of claim 1 or claim 7, wherein the functional assay comprises detecting post-translational modifications in the plurality of mistranslated variants.

24. The method of claim 1 or claim 7, wherein the functional assay comprises a protein stability assay.

25. The method of claim 1 or claim 7, wherein the functional assay comprises a measurement of protein aggregation.

26. The method of claim 2 or claim 7, wherein determining the presence of amino acid substitutions comprises identification and quantification of peptides containing the amino acid substitution and peptides not containing the amino acid substitution by mass spectrometry.

27. The method of claim 1 or claim 7, further comprising performing the method separately for each of 2 or more proteinogenic amino acid types in the target protein sequence.

28. The method of claim 1 or claim 7, where the method is performed separately for each of a plurality of amino acid types up to all 20 canonical amino acid types in the target protein sequence.

29. The method of claim 1 or claim 7, wherein the method is performed simultaneously for a plurality of different target proteins.

30. The method of claim 29, wherein the plurality of target proteins represent the proteome of a cell, or a substantial portion thereof.

31. A method of screening amino acid substitutions in a target protein for enhanced functional characteristics, comprising performing the steps recited in claim 1 or claim 7, and selecting one or more mistranslated variants that exhibit enhanced functionality compared to the target protein, and identifying the one or more substitutions in the one or more selected mistranslated variants associated with the enhanced functionality.
